(19) United States
(12) Patent Application Publication
(10) Pub. No.: US 2026/0146952 A1
(43) Pub. Date: May 28, 2026

(54) METHODS AND APPARATUSES FOR FLUOROSPECTROMETRIC MEASUREMENTS OF LOW PHOTON BUDGET SAMPLES

(71) Applicants: Ying-Ting WANG, New Taipei (TW); Matthew LEE, Conroe, TX (US)

(72) Inventors: Ying-Ting WANG, New Taipei (TW); Matthew LEE, Conroe, TX (US)

(21) Appl. No.: 19/121,997

(22) PCT Filed: Oct. 19, 2022
(86) PCT No.: PCT/US2022/078329

§ 371 Date: Apr. 17, 2025

(51) Int. Cl. G01N 21/64; G01N 21/65; G01N 33/18

(52) U.S. Cl. CPC G01N 21/6452; G01N 21/65; G01N 33/18; G01N 2201/127

(57) ABSTRACT

A method for calibrating fluorospectrometric measurements obtained on a spectrometric device is disclosed. Calibration methods of this disclosure utilizes non-fluorescent emissions of water, primarily Raman scattering signals of water, as a calibration reference. For aqueous samples containing minute amounts of fluorescent analytes, the spectrometric measurements of samples obtained will contain both fluorescent emission and non-fluorescent emission, including water Raman scattering. Calibration factors based on the water Raman peaks are provided. Spectra of samples may be compared to each other by calibrating the spectral readings against the calibration factors. Also disclosed are apparatus useful for performing the calibration methods, including lens array for enhancing the optics of spectrometric measurements and water Raman calibration methods, spectrometric devices configured to perform the calibration methods, and computer-readable medium encoding the calibration method.

### FIELD OF THE INVENTION

The invention pertains to the field of analytical chemistry. More particularly, the invention pertains to the spectroscopic analysis of fluorescence signals, methods for measurement calibration, and apparatus for performing the same.

### BACKGROUND OF THE INVENTION

Fluorescence spectra of biological samples may encode significant information about physiological or biochemical events going on in a host and holds great promise as a diagnostic tool. Databases of such spectra, when correlated to biological states, may be the foundation for metabolomics studies. For example, US Patent Publication No. US 2020/0093415A1 to Liu disclosed correlations between disease conditions and blood auto-luminescence. Lawaetz et al. also disclosed a method for early detection of colorectal cancer using fluorescence spectroscopy (Lawaetz et al., metabolomics (2012) 8: S111-S121, the entire content of which is incorporated herein by reference). However, all fluorescence-based techniques generate signals that contain not only analyte-related components, but also instrument-specific or other environmentally contributed confounding factors such as the power of the excitation source, light scattering properties of samples, the collection efficiency of the spectrometer and many other factors. These factors are generally considered background noises limiting the straightforward comparison of fluorescence data obtained, particularly in cases where the fluorescence signal is very weak (e.g. comparison on the same instrument but at different times, or measurements made on different instruments). Without a reliable method for calibrating the measurement against a common standard, the measurement data cannot be compared to each other. Therefore, the issue of calibration has been an important consideration in spectroscopic analysis.

Workman et al. discussed the overall issue of calibration and calibration transfer in spectroscopic analysis (Spectroscopy, Spectroscopy-06-01-2020, Volume 35, Issue 6, p28-32, the entire content of which is incorporated herein by reference.) One common method for signal calibration involves using serial dilution of spiked samples to establish a standard curve. Yet, when the signal of the analyte is very weak, such calibration methods may not be reliable or very difficult to achieve.

Further, trends toward big data have demanded that data sets measured on different instruments or even on the same instrument but at different time and under different measuring conditions be directly comparable. Despite efforts to understand and account for measurement variabilities, conventional calibration methodologies remain unreliable at best. The need to establish a priori a standard curve for an individual analyte is also cumbersome in practice. When a sample containing a mixture of unknown analytes in unknown combinations that gives off very weak signal, calibration methodologies based on reference samples may be quite challenging if not impossible.

Lawaetz et al. (Applied Spectroscopy, Vol 63, No. 8, p936-940, 2009, the entire content of which is incorporated herein by reference) proposed a method of calibrating fluorescence measurement using integrated area of a water Raman peak. This prior art method utilizes an empirically defined peak width for integration to obtain a Raman correction factor as a standard calibration reference for adjusting intensity variations. Lawaetz et al. proposed that this method may provide a universal Raman scale that normalizes measures to a common Raman unit, allowing measurements under different conditions and by different instruments to be compared. Lawaetz's method was nevertheless limited to samples and instrument settings that can yield good signal-to-noise ratio and relied on accurately determining the peak width. It cannot be easily generalized to calibrate for instrument biases.

Murphy disclosed a method for automatically determining the upper and lower boundaries of the Raman scatter peak in fluorescence spectroscopy (Murphy, Applied Spectroscopy, vol. 65, no. 2, p.233-235, 2011, the entire content of which is incorporated herein by reference). This method purportedly improved the accuracy of the Raman unit method, but still does not address the limitations of unknown confounding factors that may distort the spectra. In cases where the concentration of the biological sample is very small, leading to very low photo budget of the corresponding fluorescence emission, the sample spectra may be significantly complicated by various background signals, including the Raman signals. Moreover, if the quantification involves the intensity ratio between two different fluorophores excited at different wavelength, machine-dependent calibration may be required. Murphy's method does not account for such scenario.

Therefore, there still exists a need for better calibration methods that can be generalized to correct for instrument biases and batch biases in spectrometric measurements.

### SUMMARY OF THE INVENTION

Accordingly, in a first aspect, the present invention provides a method for calibrating fluorospectrometric measurements of a test sample taken on a spectrometric device using non-fluorescent emissions of water as a calibration reference. Methods in accordance with this aspect of the present invention will generally include the steps of correcting for background noises in the fluorospectrometric measurements of the test sample by removing non-fluorescent emission signals from the measurements; and calibrating the corrected fluorospectrometric measurements with one or more calibration factors that relate non-fluorescent emission peaks of water in the test sample to the corresponding non-fluorescent emission peaks of water in a reference sample.

Fluorospectrometric measurements are typically obtained by first directing an excitation light on the test sample, and then detecting emission light coming out of the test sample. In some embodiments, fluorospectrometric measurements are spectral readings encompassing a range of emission wavelengths. In some other embodiments, fluorospectrometric measurements are readings of discrete emission wavelengths at one or more predetermined wavelengths.

The excitation light is preferably monochromatic light having a predetermined wavelength. In some embodiments, a single wavelength is used. In some other embodiments, a plurality of wavelengths is used. In some preferred embodiments, a wavelength range having a predetermined increment between each discrete wavelength is employed.

Numerous types of light sources are known in the art and are not particularly limited herein. Exemplary light sources may include light-emitting diodes (LEDs) and laser diodes, mercury or xenon arc-lamps, and halogen lamps, or any other light sources known in the art. It is understood by those skilled in the art that each light source differs in the way wavelengths of light are delivered, which may influence instrument design, optical analysis and the readings will have to be appropriately corrected for background noise and calibrated for experimental and instrumental variabilities.

Spectrometric devices usable with methods of the present invention are not particularly limited. They may be a filter fluorometer that uses light filters to isolate lights of particular wavelengths or a grating type that uses diffraction grating monochromators to isolate the incident light and fluorescent light.

Non-fluorescent emissions of water may include Raman scattering, Rayleigh scattering, and any other non-fluorescent light emissions coming from water after being excited by the excitation light. In some preferred embodiments, the non-fluorescent emission includes only Raman scattering. In some other preferred embodiments, the non-fluorescent emission includes both Raman scattering and Rayleigh scattering.

Test samples suitable to be measured and calibrated by methods of the present invention are generally aqueous samples. In some embodiments, the test sample contain one or more fluorescent analytes in minute quantity, preferably in the concentration range of (picomolar (pM) to micromolar (μM); more preferably from about 10 pM to about 10 μM. In some preferred embodiments, the test sample may be a biological sample. Exemplary biological sample may include whole blood samples, blood serum samples, aqueous samples containing flavonoids, saliva, urine, sputum, or a combination thereof.

Fluorospectrometric measurements of test samples should be corrected for background noise before calibration. The background noises to be corrected include Raman scattering, Rayleigh scattering, environment light, and thermal noise of devices.

In some embodiments, Raman scattering and Rayleigh scattering are both removed from the fluorospectrometric measurements. In other embodiments, only Raman scattering signals are removed from the fluorospectrometric measurements.

Calibration factors of the present invention are formulated to scale measurements of the test sample to the same scale as that of the reference sample. These factors mathematically transform one set of measurements to another so that variabilities due to instrumentation, environmental factors and other variables may be normalized.

Any number of input parameters and mathematical relations may be advantageously employed to formulate the calibration factor. In some embodiments, the calibration factor is a ratio between the peak height of water Raman of the reference sample and the test sample. In some other embodiments, the calibration factor may be the ratio between the area under the water Raman peak of the reference sample and the test sample. In still some other embodiments, a combination of peak height and area under the curve may be used.

Reference sample usable for calibration purposes may be a water blank or an aqueous sample containing one or more fluorescent analytes.

In those embodiments where a water blank is used as the reference sample, all other samples measured on the same device with the same instrument settings can be calibrated against the blank so that the results may be compared on the same calibrated scale.

In those embodiments where no water blank is used, non-fluorescent emissions of the samples to be compared may serve as an internal reference. When comparing measurements of one sample to another, the non-fluorescent emission of one sample can serve as the reference for the second sample to calibrate against.

The wavelength of the excitation light may be the same for both fluorescent and non-fluorescent emissions. The emission wavelengths of water Raman scattering and Rayleigh scattering are generally well-understood and predictable. Test samples such as blood samples that contain biological molecules may have very distinct spectral peaks separate from those of water Raman and Rayleigh scattering. Thus, calibration may be performed using non-fluorescent emissions generated by the same excitation light. However, in some cases, the fluorescent signals of a test sample may overlap with the non-fluorescent signals of its water solvent. In such cases, it may be desirable to use a different excitation wavelength to generate non-fluorescent emission peaks that can be clearly distinguished from the fluorescent peaks. Accordingly, in some embodiments, a step of real-time “peak seeking” may be included. Specifically, in some embodiments, methods in accordance with this aspect of the invention may further include the steps of dynamically changing the wavelength of the excitation light directed at the test sample until the Raman peak is clearly distinguished from the rest of the spectra.

In a second aspect, the present invention provides a spectrometric device configured to perform a calibration method as described above in the first aspect.

In some embodiments, the spectrometric device is configured as a plate reader. In some other embodiments, the spectrometric device is configured to receive a cuvette for single sample analysis. In still some further embodiments, the spectrometric device also includes a control unit for controlling the operation of the device, a memory unit for recording spectrometric measurements and computer-readable instructions that encode instructions for performing a calibration method as described in the first aspect above, and a computing unit for executing the computer-readable instructions and processing the spectrometric data.

In a third aspect, the present invention provides a computer-implemented method for analyzing spectrometric data. Methods in accordance with this aspect of the invention will generally include computer-readable instructions to perform a method of the first aspect.

In a fourth aspect, the present invention provides a computer-readable medium encoding methods in accordance with the third aspect of the invention as described above.

In a fifth aspect, the present invention provides a lens-array cap for use with a multi-well container useful for fluorospectrometric analysis. Lens-array caps in accordance with this aspect of the invention, will generally have a transparent base plate with a plurality of lenses arranged on the base plate.

Multi-well container suitable for use with this aspect of the invention will generally have a plurality of wells formed on a transparent plate, each having an upward-facing opening with a predetermined well depth. The material and size of the containers are not particularly limited so long as the material is transparent to the excitation and emission light.

The plurality of lenses is arranged on the base plate such that when applied to a matching multi-well container, each lens of the lens array will completely cover one well opening. Each lens is also configured such that when an excitation light goes through the lens, the light beam will be focused at about the center of the well.

In some embodiments, the lens array cap is configured as a stand-alone unit to be used with other multi-well containers. In some other embodiments, the lens array and the multi-well container form an integrated unit. In one exemplary embodiment, the lens array may be configured as a hinged cover of the multi-well container.

It will be appreciated by those skilled in the art that while the fifth and sixth aspects of the invention are described in terms of containers having multiple wells and container caps having multiple matching lenes, single well containers may also benefit from a lens cap configured to focus excitation light at about the center of the container to maximize emission light yield. Thus, the fifth and sixth aspects of the invention also includes single well containers such as cuvettes with a matching single lens cap configured to completely cover the upward-facing well opening.

In some embodiments, the analytical containers may have an elongated tubular body having a hollow well for holding a sample therein. In such embodiments, the lens cap may be implemented by the cross-sectional geometry of the container. Exemplary cross-sectional geometry may be a circular cross-section with a plannar side such that the curvature on the plannar side form a convex lens capable of focusing incoming light at the center of the container's cross-section. In some preferred embodiment, the cross-section has a regular geometric shape. Exemplary geometric shape may include but not limited to circular, triangular, rectangular, pentagonal, hexagonal, and other suitable geometric shapes.

In some embodiments, the analytical container may be a tube for conducting fluidic sample flowing therethrough. In these embodiments, the tube may be configured with a cross-section having an optically-enhanced side capable of focusing incoming line at the center of the tube's cross-section. In some further embodiments, the tube may be a blood tube for an extracorporeal circuit. In still some further embodiments, the tube may optionally include a sample reservoir, wherein the sample reservoir may further have an optically-enhanced window capable of focusing incoming light at the center of the reservoir. A spectrometric measuring device may be advantageously adapted to such a tube and perform in situ measurement to perform continuous, semi-continuous, or ad hoc measurement of samples flowing therethrough.

Other aspects and advantages of the invention will be apparent from the following description and the appended claims.

### BRIEF DESCRIPTION OF THE DRAWINGS

FIG. 1 shows an exemplary workflow of a Raman calibration method in accordance with embodiments of the present invention.

FIG. 2 shows a schematic diagram for an exemplary spectrometric device in accordance with embodiments of the present invention.

FIG. 3 shows another schematic diagram for another exemplary spectrometric device in accordance with embodiments of the present invention.

FIG. 4 shows an exemplary implementation of a lens array cap in accordance with embodiments of the present invention.

FIG. 5 shows an exemplary implementation of an optically-enhanced analytical container in accordance with embodiments of the present invention.

FIG. 6 shows an exemplary implementation of an extracorporeal circuit with an optical window for fluorescence measurement in accordance with embodiments of the present invention.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be clear and apparent to those skilled in the art that the subject technology is not limited to the specific details set forth herein and may be practiced without these specific details. In some instances, well-known structures and components are shown in block diagram form in order to avoid obscuring the concepts of the subject technology.

For purposes of this specification, the term “fluorospectrometric device” is used interchangeably with fluorospectrometer, fluorescence spectrophotometer, fluorometer, or fluorescence spectrometer. Such devices perform the general function of directing an excitation light at a test sample (typically in a sample-holding vessel or container) and detecting the emitted light which can either be resulting from fluorescent excitation of fluorophores in the sample or non-fluorescent scattered light such as Raman scattering emission.

The term “light” as used in this specification refers to both non-visible light (e.g., ultraviolet light) and visible light, that is, light visible to the naked eye.

### Raman and Rayleigh Scattering

When a high intensity monochromatic light source, such as that provided by a laser, is directed onto an analyte molecule (or sample), a majority of the incident photons are scattered by the analyte molecule elastically, meaning that the scattered photons have the same energy (and, therefore, the same frequency) as the incident photons. This elastic scattering is termed Rayleigh scattering, and the elastically scattered photons and radiation are termed Rayleigh photons and Rayleigh radiation, respectively. However, a small fraction of the photons (e.g., about 1 in 107 photons) are inelastically scattered by the analyte molecule. These inelastically scattered photons have a different frequency than the incident photons. The inelastic scattering of photons is termed the Raman effect. The inelastically scattered photons may have frequencies greater than, or, more typically, less than the frequency of the incident photons.

When an incident photon collides with a molecule, energy may be transferred from the photon to the molecule or from the molecule to the photon. When energy is transferred from the photon to the molecule, the scattered photon will emerge from the sample having a lower energy and a corresponding lower frequency. These lower-energy Raman scattered photons are commonly referred to in Raman spectroscopy as the Stokes radiation. A small fraction of the analyte molecules is already in an energetically excited state. When an incident photon collides with an excited molecule, energy may be transferred from the molecule to the photon, which will emerge from the sample having a higher energy and a corresponding higher frequency. These higher-energy Raman scattered photons are commonly referred to in Raman spectroscopy as the anti-Stokes radiation.

The Stokes and the anti-Stokes radiation is detected by a detector, such as a photomultiplier or a wavelength-dispersive spectrometer, which converts the energy of the impinging photons into an electrical signal. The characteristics of the electrical signal are at least partially a function of the energy (or wavelength, frequency, wave number, etc.) of the impinging photons and the number of the impinging photons per unit time (intensity). The electrical signal generated by the detector can be used to produce a spectral graph of intensity as a function of frequency for the detected Raman signal (i.e., the Stokes and anti-Stokes radiation). A unique Raman spectrum corresponding to the particular analyte may be obtained by plotting the intensity of the inelastically scattered Raman photons against their frequency or, equivalently and more commonly, their wavenumber in units of inverse centimeters. A Raman spectrum readout is often presented as a plot of intensity versus Raman shift, the Raman shift being defined as a difference between the wavenumbers of the source radiation (excitation radiation) and the Raman-scattered radiation. Peaks and valleys that are meaningful for purposes of chemical analysis are typically for Raman shifts in the range of 500 cm⁻¹-2000 cm⁻¹, which for a typical source wavelength of 1000 nm would correspond to Raman-scattered photons having wavelengths between 1050 nm to 1250 nm.

This unique Raman spectrum has been used for many purposes such as identifying an analyte, identifying chemical states or bonding of atoms and molecules in the analyte, and determining physical and chemical properties of the analyte. Raman spectroscopy may be used to analyze a single molecular species or mixtures of different molecular species. Furthermore, Raman spectroscopy may be performed on a number of different types of molecular configurations, such as organic and inorganic molecules in either crystalline or amorphous states.

In the context of the present invention, one exemplary signal of interest is the fluorescence emission of chemical species in a biological sample such as a subject's blood or blood plasma. Fluorescence spectroscopy is the re-emission of an incident photo following its absorption by an analyte molecule. Raman scattering and fluorescence are two fundamentally different but competing phenomena. Thus, when obtaining a Raman spectrum, a Raman spectroscopist generally aims to avoid generating fluorescence signals and vice versa.

Raman Spectra as Internal References for Samples with Low Fluorescence Intensity

Before raw fluorescence spectral data can be used for analysis, several types of data correction should be performed to minimize measurement biases, including correction for instrument biases, sample absorption biases (often referred to as inner filter effect), batch biases, background removal and, intensity calibration. Correction for instrument biases addresses the issue of peak position shifting due to instrument variabilities. Correction for sample absorption biases addresses the issue of emission signal attenuation due to the length of the optical path and the concentration of analyte in the sample. This phenomenon is often referred to as the inner filter effect. In optically thin samples with low absorbance, this is not an issue so correction for inner effect may not be necessary. On the other hand, spectral position, batch variation, and emission intensity readings are all highly dependent on the instrument. For example, different instrument may use different detectors which translates to different measurement scales. Batches measured at different time, even on the same instrument and same setting may have different intensity reading due to factors such as power fluctuation. Also, intensity readings are generally given in arbitrary units (except for photon counting systems), which makes direct quantitative comparison of data across instruments difficult if not impossible.

Prior art approaches to the aforementioned problems generally rely on the use of a serially diluted calibration standard. When working with well characterized and known fluorophores, this approach works well. However, when working with complex mixture of potentially unknown fluorophores, this approach does not work. To date, most prior art studies attempt to avoid this problem by either carrying out all measurements on the same instrument or by using an external well-characterized standard such as quinine sulfate or standard material 2941 (supplied by NIST).

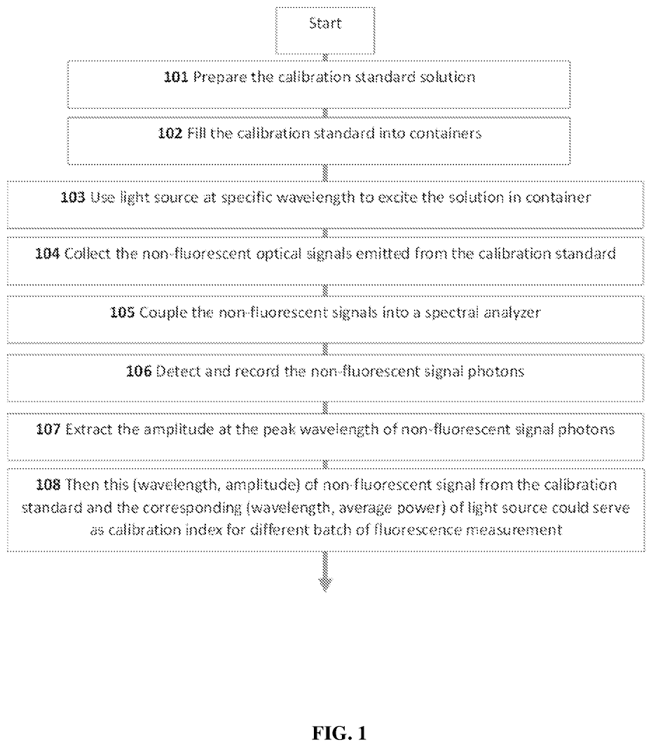

Disclosed herein is a general method of correcting fluorescence spectral data that takes into account biases due to instrument variation, sample-to-sample (batch) variation, and intensity scale variations. Methods in accordance with embodiments of the present invention are particularly suited for samples containing trace amounts of fluorescent analyte that yield fluorescent signals in about the same dynamic range as that of water Raman scattering. FIG. 1 outlines an exemplary workflow of performing calibration with Raman spectra in a spectral analysis.

Referring to FIG. 1, step 101, “Prep Calibration Solution”, a calibration solution is first prepared. In some embodiments, the calibration solution is simply a water blank. In other exemplary embodiments, solvents of test samples possessing characteristic Raman scattering properties may also advantageously be used as the calibration solution. In other exemplary embodiments, the Raman scattering spectra of a solvent may further be used both as the calibration solution and the internal control of fluorescence spectroscopic measurements. In some preferred embodiments, the dominant solvent of the test sample is water.

Compared with conventional fluorescence-based or absorption-based calibration methods, calibration method in accordance with methods disclosed herein will not have the problems of photo-bleaching or limitation on the excitation wavelength. Moreover, preparation of the calibration solution requires less quality control on the storage or powder dissolution.

Next, in step 102, “Load Calibration Solution”, the calibration solutions are loaded into suitable analytic containers. Those skilled in the art will appreciate that this step is optional for embodiments in which a blank is not used. It will also be understood by those skilled in the art that analytic containers suitable for use in connection with methods of the present invention are generally designed for spectroscopy analysis. They are configured such that the excitation light could uniformly illuminate the sample and the emitted Raman scattering signals could be efficiently collected.

In some embodiments, to avoid extra attenuation, light-passing walls of the analytic containers should be optically transparent and flat.

In some embodiments, to avoid background interference, the analytic containers are preferably constructed from materials that emit no auto-fluorescence in the excitation range of 340-650 nm.

Solvents suitable for use as calibration solution in connection with methods of the present disclosure preferably has stable, unique and robust Raman scattering properties. The calibration solution could be pre-loaded or manually loaded into the analytic containers. In some preferred embodiments, suitable containers are loaded with the calibration standards.

In step 103, “Generate Calibration Signals”, a light source having a predetermined wavelength is directed at the analytic containers to excite the calibration solution inside the container. For each excitation wavelength used in the fluorescence measurement, the Raman scattering signals of the calibration solution at that specific excitation wavelength should be excited and measured. For example, if 488 nm light is used to excite flavin fluorescence in blood, then excitation of the calibration solution at 488 nm should also be measured.

Compared with conventional calibration methods, Raman scattering-based calibration methods disclosed herein have more flexibility on the choice of excitation wavelength. When the excitation light source incidents upon the calibration solution, light will be emitted. The emitted light may have a fluorescent and a non-fluorescent component.

In step 104, “Collect Calibration Signals”, the non-fluorescent optical signals emitted from the calibration solution are collected. When the fluorescence signal of the test sample is weak, other signals such as Raman scattering, Rayleigh scattering, and even Mie scattering of solvents may interfere with the test sample fluorescence spectra. By measuring the Raman scattering, Rayleigh scattering, and even Mie scattering signals of solvents, one not only can remove the background interference in the fluorescence spectra, but also find the batch-to-batch variation ratio of excitation/collection efficiency in the measurement system. Accordingly, in this step, the non-fluorescent optical signals is collected via a light collection element for further analysis. Exemplary light collection elements may include lens to collimate diverge signals, lens to focus signals into the entrance slit of a spectrometer system, a dichroic beam splitter to separate the directions of excitation and signal collection, a notch filter to block the stray excitation light, a long pass or band pass filters to further block environment background, and other suitable light collection devices known in the art.

In step 105, “Couple Calibration Signals”, the non-fluorescent signals are coupled into a spectral analyzer. Similar to fluorescence, spontaneous Raman scattering signals could also emit in omni direction, including the 90-degree side-collection direction or the backward-collection direction. Recognizing that the excitation and emission wavelength of Raman scattering is close to the fluorophores of interest, the batch-to-batch variation ratio of excitation/collection efficiency in the measurement system could be constantly monitored at the same system setting. Accordingly, the non-fluorescent signals collected are operatively coupled into a spectral analyzer to normalize the batch-to-batch signal variation. Exemplary spectral analyzer may include a grating or prism to disperse color of light into different propagation directions, a lens or curve mirrors for the focusing of each color ray onto the defined point of detection, and a photoelectric detector system with one pixel or arrays of pixels. The photoelectric detector system could be photomultiplier tubes (PMT), charge-coupled device (CCD), or avalanche photodiodes (APD). Coupling of the non-fluorescent signals may be achieved by a lens to collimate diverge signals, a dichroic beam splitter to separate the directions of excitation and signal collection, a notch filter to block the stray excitation light, a long pass or band pass filters to further block environment background, and then a lens to focus signals into the entrance slit of a spectrometer system.

In step 106, “Detect Calibration Signals”, the non-fluorescent signal photons are detected and recorded. After coupling the non-fluorescent signals into the spectrometer, the Raman scattering spectra of the calibration solvent can then be recorded and measured by the spectrometer. One should be mindful that aside from Raman scattering, Rayleigh scattering of the excitation source could also bleed through into the fluorescence detection range due to stray light or poor resolution of the spectrometer. These spectral interference can be measured through the excitation of the standard solvent.

In step 107, “Extract Calibration Spectral Features”, the amplitude and shape of the Raman and Rayleigh scattering spectrum are extracted. Methods for extracting the spectral features are known in the art. Exemplary extraction methods may include major component analysis on the spectrum, change the excitation wavelength and identify it by the shift of Raman peak wavelength, not limited thereto. For example, considering that the Raman scattering spectra has a similar shape as the excitation source. By amplitude fitting, one could extract the Raman spectra and its amplitude from the Rayleigh background resulting from stray light or the poor resolution of spectrometer.

In some embodiments, identification of the Raman peaks may be achieved by visual inspection of the emission spectrogram. In some other embodiments, Raman peaks may be identified computationally using computer-implemented methods. In a preferred embodiment, the Raman peaks is identified by computing a first-order derivative curve of the emission spectrogram with respect to emission wavelength, and then locating the emission wavelength at which the derivative curve crosses over the zero point. The derivative curve is taken at the wavelength range where the Raman peak is expected to be. In some preferred embodiment, the range is from about 400 nm to about 600 nm.

In some other embodiments, Raman peak identification is performed in real-time. In these embodiments, the excitation wavelength is varied, and the emission spectrum is observed for changes of peak location so as to verify the identity of the peak. It will be appreciated by those skilled in the art that in situations where the analyte's fluorescence emission peaks overlap those of the water Raman at a given excitation wavelength, changing the excitation wavelength may result in a different emission profile that separates water Raman peaks from the analyte's fluorescence emission peaks, thereby, providing a better signal-to-ratio profile.

In step 108, “Determine Calibration Parameters”, the extracted amplitudes of Raman scattering signals are divided into reference values to obtain calibration ratios for fluorescence dosimetry. The (wavelength, amplitude) value of the non-fluorescent signal from the calibration standard and the corresponding (wavelength, average power) value of the light source may serve as a calibration index for evaluating the batch-to-batch variation ratio of excitation/collection efficiency in the fluorescence spectra measurement system.

### Fluorescent Spectroscopy System Design

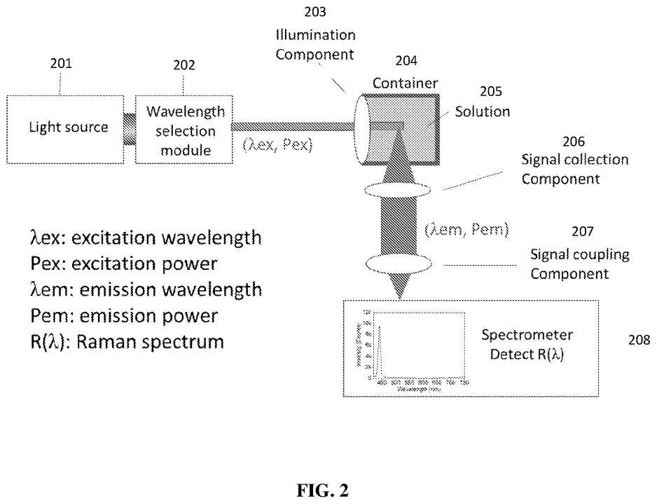

FIG. 2 shows schematics of a fluorescent spectroscopy system implementing methods disclosed herein. Referring to FIG. 2, systems implementing methods disclosed herein will generally include one or more excitation light source(s) 201, one or more wavelength selection module(s) 202, one or more illumination component 203, analytic containers 204 for holding liquid analyte, sample solution 205, signal collection component(s) 206, signal coupling component(s) 207, and the spectrometer 208.

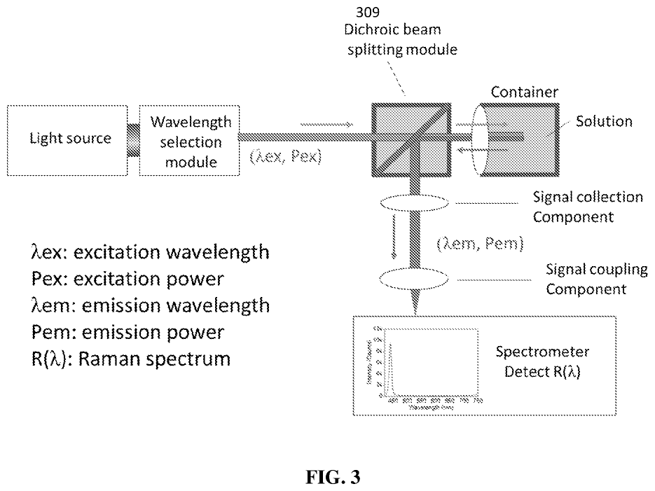

For the backward collection scheme, the illumination and signal collection component will be the same, but the signal will be separated from excitation by a dichroic beam splitter 309 (see FIG. 3).

### Signal Collection Component Design

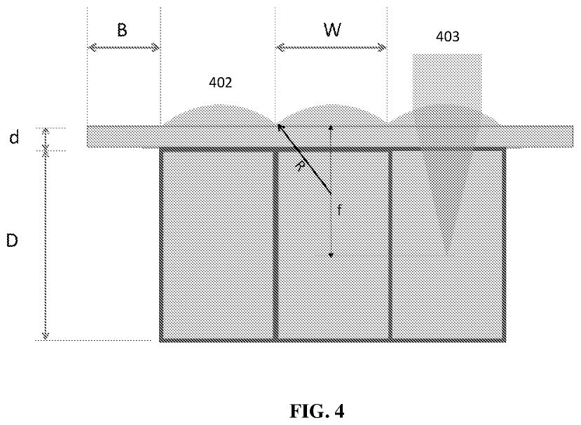

Referring to FIG. 4, an exemplary design suitable for illumination and collection of non-fluorescent signal is shown. In some embodiments, the light illumination component 203 and collection component 206 may be configured to form an integrated illumination/collection component (203/206) together with the analytic container 204. In some exemplary embodiments, the illumination/collection components are micro-lenses 402 arranged into a lens array 403 disposed on a substrate 404 having a thickness d. On the edge of the substrate, a boarder having width B may be reserved. The analytic container 204 having length D. In use, the substrate may be capped over the analytic container 204 to act as the illumination face of the container. Advantageously, each of the micro-lens 402 is configured with a radius of curvature R such that the effective focal distance f with solution analyte will be located within the analyte containing space D of the analytical container. This way, the lenses will focus the illumination light 403 on the analyte solution to effectively generate and collect the weak Raman signals. In such embodiments, the illumination and signal collection component are integrated as one component with the container.

### Optically-enhanced Analytical Containers and Extracorporeal Circuit Tubes

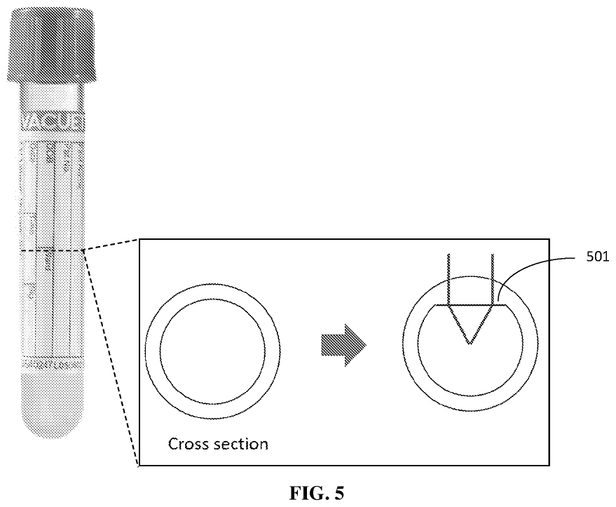

Conventional sample containers such as blood sample tubes and cuvettes shown in FIG. 5 may be configured with an optically-enhanced window 501 for directing line to focus at the center of the container to obtain a favorable Raman signal. While FIG. 5 shows an exemplary cylindrical blood collection tube adapted with an enhanced optical window, it will be understood by those skilled in the art that the tube does not have to be cylindrical in shape. It may have a rectangular cross-section as in the case of a cuvette or other cross-section geometry.

In addition to stand-alone analytical sample containers described above, the enhanced optical window may also be advantageously implemented on an extracorporeal tube so that samples may be contiguously measured by a spectrometric device.

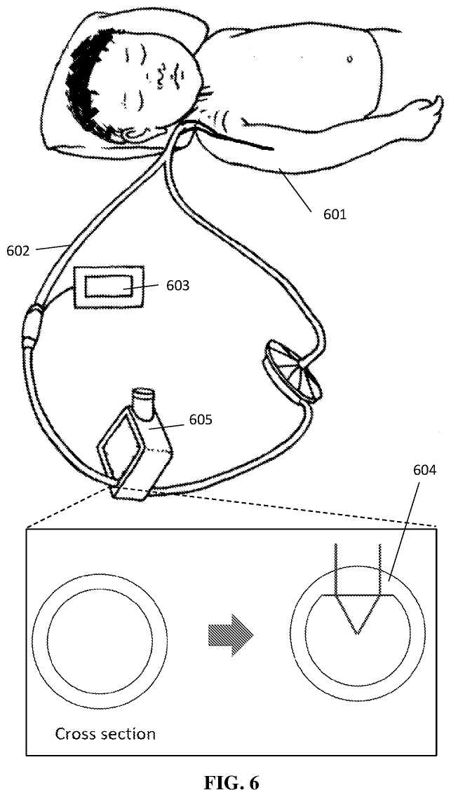

Referring to FIG. 6, a human subject 601 is operatively connected to an extracorporeal circuit tube 602. In some embodiments, the tube may optionally be configured with a sampling reservoir 603 which may have a window configured with an optical lens for focusing excitation light at the center of the reservoir. In some other embodiments, the extracorporeal circuit tube may be configured to have a cross-section 604 that function as an optical lense capable of focusing incoming excitation light at the center of tube. In such embodiments, a spectrometric measurement device 605 may be adapted to continuously monitor fluorescence of the sample fluid in the extracorporeal circuit.

To further illustrate the present invention, the following specific examples are provided.

### Example 1

### Calibration of Fluorescent Spectroscopic Measurements Using Raman Spectra

The following outlines a general experimental protocol for obtaining fluorescent spectrum of a blood sample that is normalized using the Raman calibration method disclosed herein.
1. Prepare 1 mL deionized water and use a 200 nm pore size filter to remove the debris in water. This solution serves as the calibration standard.
2. Fill the calibration standard solution into a cuvette having 1 cm×1 cm cross-section area.
3. Insert the cuvette with the calibration standard solution into the spectrometer.
4. Turn on the Xe lamp and select the excitation wavelength by a monochromator. The central wavelength of each excitation band could be λ1=340 nm, λ2=385 nm, λ3=405 nm, λ4=460 nm, λ5-488 nm, λ6=530 nm, and λ7=580 nm. Each wavelength can efficiently excite certain fluorophores in a blood sample.
5. Tune the output slit size of the monochromator such that the excitation bandwidth of each excitation band is 1 nm. The shape of the spectra could be modeled by a Gaussian function.
6. Measure the output power of each band after the output of monochromator.
7. The excitation light is focused into the cuvette and the generated signal is collected at the direction perpendicular to the excitation axis.
8. Couple the signal into a spectrometer with 200 nm entrance slit size.
9. The detector in the spectrometer is cooled down to about-30 degree to suppress the thermal noise.
10. By rotating the grating, the intensity of the Raman spectra is recorded at a 1 nm spectral resolution. Each data point has an 0.5 second integration time. The data is the output voltage from the transimpedance amplifier of the sensitive detector.
11. Measure the amplitude of the Raman peak at different excitation band.
12. Divide these Raman amplitude at different wavelength into those base value measured at the base day of experiment to obtain a calibration coefficient. This coefficient is referred to as the normalization ratio of the fluorescence spectra.
13. Then perform the measurement of blood sample's fluorescence spectra. Fill the 1 mL blood sample into the cuvette, sweep the excitation bands from λ1 to λ7, and measure the correspond fluorescence spectra. The excitation and detection conditions are the same as those of Raman measurement.
14. The non-fluorescent background signals (Raman peak and Rayleigh tail) in fluorescence measurement could be removed by a fractional subtraction.
15. To fairly compare the peak intensity of blood fluorescence spectra measured at that day to that measured at the base day, we need to divide the value of fluorescence spectra by the normalization ratio.

$${F}_{2} ( λ ) = [ {M}_{s 2} ( λ ) - a \times W {R}_{s 2} ( λ ) - b \times R {T}_{s 2} ( λ ) ] \times S \times B \times M$$

wherein:
F₂(λ) is the fluorescence spectra of sample 2 under specific excitation wavelength, excitation bandwidth, and excitation power;
Mₛ₂(λ) is the measured emission spectra of sample 2;
WRₛ₂(λ) is the Water Raman scattering spectra of sample 2;
RTₛ₂(λ) is Rayleigh tail spectra of sample 2

$$S = \frac{W {R}_{S 1} ( λ )}{W {R}_{S 2} ( λ )}$$

is the sample-to-sample calibration factor

$$B = \frac{W {R}_{b 1} ( λ )}{W {R}_{b 2} ( λ )}$$

is the batch-to-batch calibration factor

$$M = \frac{W {R}_{m 1} ( λ )}{W {R}_{m 2} ( λ )}$$

is the machine-to-machine calibration factor

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

What is claimed is:

1. A method for calibrating fluorospectrometric measurements of a test sample taken on a spectrometric device, comprising:
removing non-fluorescent emission signals from the fluorospectrometric measurements; and
calibrating the fluorospectrometric measurements with one or more calibration factors that relate non-fluorescent emission peaks of water in the sample to the corresponding non-fluorescent emission peaks of water in a reference sample.

2. The method of claim 1, wherein the sample contains one or more fluorescent analytes in the range of from about 10 pM to about 10 μM.

3. The method of claim 1, wherein the reference sample is a water blank.

4. The method of claim 1, wherein the calibration factor is a ratio of the reference sample's water Raman peak height and the test sample's water Raman peak height.

5. The method of claim 2 further includes selecting a water Raman peak for the test sample by varying the excitation wavelength and comparing the associated water Raman peak.

6. A spectrometric device configured to automatically calibrate fluorospectrometric measurements using water Raman peaks, comprising:
a light source for providing an excitation light;
a detector for detecting emission light and recording spectrometric measurements; and
a data processing unit configured to perform the method of claim 1.

7. The spectrometric device of claim 6, wherein said device is configured as a plate reader.

8. A lens array for use with a microtiter plate readable by a spectrometric device, wherein said microtiter plate has a plurality of wells each having an upward-facing well opening with a predetermined well depth, said lens array comprising:
a plurality of lens elements arranged as an array to match the microtiter plate well openings such that when the lens array is placed on top of the microtiter plate each of the lens element completely covers one well opening; and that each lens element is configured to focus light at the center of the well.

9. A spectrometric sample container, comprising:
a substrate having a plurality of wells each configured for retaining an aqueous sample; and
the lens array according to claim 8.

10. A spectrometric sample container for use with the spectrometric device of claim 6, comprising:
an elongated tubular body having an optically-enhanced cross-section configured to function as a light focusing lens to focus an incoming light incident upon the optically-enhanced cross-section at the center of the cross-section.

11. The container of claim 10 wherein the elongated tubular body has a regular geometric cross-section with one side configured to function as the light focusing lens.

12. A spectrometric sample container for use with the spectrometric device of claim 6, comprising:
a tube for conducting a fluidic sample to flow therethrough, wherein said tube is configured to have a cross-section having an optically enhanced side capable of focusing incoming light at the center of the tube's cross-section.

13. The container of claim 12, further comprising:
a sample reservoir having an optically enhanced window configured to focus incoming light a the center of the sample reservoir.

14. A computer-readable medium encoding method for analyzing spectrometric data, comprising:
a persistent digital data storage medium readable by a computing unit encoding instructions for performing the method of claim 1.
